(19) United States
(12) Patent Application Publication
(10) Pub. No.: US 2021/0235997 A1
(43) Pub. Date: Aug. 5, 2021

(54) FLAGGING A PORTION OF A RECORDING FOR REVIEW

(71) Applicant: KONINKLIJKE PHILIPS N.V., EINDHOVEN (NL)

(72) Inventors: Elise Claude Valentine TALGORN, Eindhoven (NL); Lucas Jacobus Franciscus GEURTS, Sterksel (NL); Sharon WILLIAMS, Eindhoven (NL); Jasper de Vreede, Eindhoven (NL); Niels LAUTE, VENLO (NL); Rob van Gansewinkel, Eindhoven (NL)

(21) Appl. No.: 17/049,409

(22) PCT Filed: Apr. 29, 2019
(86) PCT No.: PCT/EP2019/060877

§ 371 Date: Oct. 21, 2020

Related U.S. Application Data
(60) Provisional application No. 62/664,375, filed on Apr. 30, 2018.

(51) Int. Cl. A61B 5/0205; A61B 5/11; A61B 5/024; A61B 5/145; A61B 5/0531; A61B 5/369; A61B 5/389; A61B 5/318; A61B 5/16; A61B 5/00; A61B 5/1171; G16H 10/60; G10L 15/22; G10L 15/18

(52) U.S. Cl. CPC A61B 5/0205; G10L 15/1815; A61B 5/024; A61B 5/14546; A61B 5/0531; A61B 5/369; A61B 5/389; A61B 5/318; A61B 5/163; A61B 5/4803; A61B 5/1176; A61B 5/165; A61B 5/746; G16H 10/60; G10L 15/22; A61B 5/1116

(57) ABSTRACT

Computer implemented methods and systems of flagging a portion of a recording of a patient interaction with a clinical professional for review. A method comprises acquiring a recording of a patient interaction with a clinical professional, acquiring physical data relating to a physical state of the patient during the recording, and based on the physical data, flagging a portion of the recording for review.

### FIELD OF THE INVENTION

Embodiments herein relate to flagging a portion of a recording of a patient interaction with a clinical professional for review. More specifically, but non-exclusively, some embodiments herein relate to flagging a portion of a recording of a patient interaction for review, based on physical data relating to the physical state of the patient during the recording.

### BACKGROUND OF THE INVENTION

The general background is in the conveyance of information from a clinical professional to a patient. The general background is furthermore in personal computing assistants.

During medical consultations where a serious diagnostic like cancer is given to a patient by a clinical professional, the patient may be overwhelmed, both by the implications of the diagnosis itself and by the volume of information provided to the patient by the clinical professional. As a result, patients may not be capable of fully listening to what the clinical professional tells them. As a result, patients may not have access to the information they need to understand the diagnosis or make appropriate decisions relating to their care.

There is thus a need to improve patient understanding of information conveyed by clinical professionals, particularly when distressing news is relayed.

### SUMMARY OF THE INVENTION

As noted above, patients facing bad news such as a cancer diagnosis may be distracted and unable to process information provided to them. If the clinical professional notices that the patient is not taking in the information being given to them, they can ask if the patient understands what they are being told, and they may then repeat some or all of the information.

However, sometimes there may be so much information that the clinical professional cannot know exactly what has been taken in and what needs repeating. Furthermore, clinical professionals often have a limited amount of time in consultation appointments and may not be able to repeat the information as many times as may be needed. Finally, even if a clinical professional were able to repeat the information a limitless number of times, the patient may be in such shock that they may not be able to fully assimilate the information during the appointment.

Thus according to a first aspect, there is provided a computer implemented method comprising acquiring a recording of a patient interaction with a clinical professional, acquiring physical data relating to a physical state of the patient during the recording, and based on the physical data, flagging a portion of the recording for review.

In this way, a patient interaction may be recorded and certain segments of the recording may be flagged for review, based on the patient's physical (and thus emotional) state.

According to a second aspect, there is provided a computer program product comprising a non-transitory computer readable medium, the computer readable medium having computer readable code embodied therein, the computer readable code being configured such that, on execution by a suitable computer or processor, the computer or processor is caused to perform the method of the first aspect.

According to a third aspect, there is provided a system comprising a memory comprising instruction data representing a set of instructions and a processor configured to communicate with the memory and to execute the set of instructions. The set of instructions, when executed by the processor, cause the processor to acquire a recording of a patient interaction with a clinical professional, acquire physical data relating to a physical state of the patient during the recording, and based on the physical data, flag a portion of the recording for review.

In this way, a patient may be supported after receiving bad news about their health in a consultation with a clinical professional. Information that may have been missed by the patient, or information that the patient is not ready to receive during the consultation may be detected and flagged for review at a later time.

These and other aspects of the invention will be apparent from and elucidated with reference to the embodiments described hereinafter.

### BRIEF DESCRIPTION OF THE DRAWINGS

For a better understanding and to show more clearly how embodiments herein may be carried into effect, reference will now be made, by way of example only, to the accompanying drawings, in which:

FIG. 1 is a flowchart of an example method of flagging a portion of a recording of a patient interaction for review according to some embodiments;

FIG. 2 shows an example method of flagging a portion of a recording of a patient interaction for review according to an embodiment;

FIG. 3 shows an example system for flagging a portion of a recording of a patient interaction for review according to some embodiments.

### DETAILED DESCRIPTION OF EMBODIMENTS

As described above, embodiments herein are aimed at improving a patient's understanding of information relayed to them by a clinical professional. Patients may, for example, be distracted, distressed or otherwise unable to assimilate information provided by a clinical professional during a medical consultation, particularly following the receipt of bad news.

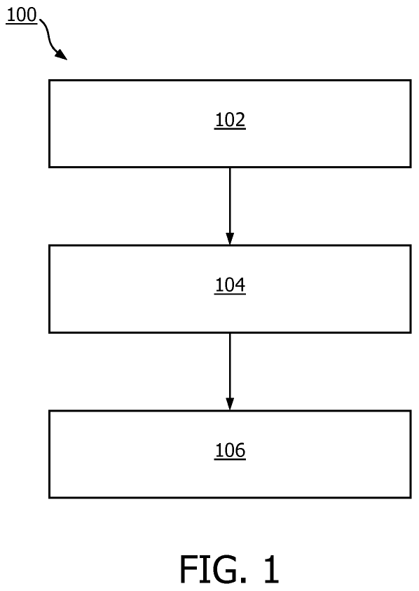

FIG. 1 illustrates a computer implemented method 100 according to embodiments herein. The computer implemented method comprises, in a block 102, acquiring a recording of a patient interaction with a clinical professional, in a block 104 acquiring physical data relating to a physical state of the patient during the recording and in a block 106, based on the physical data, flagging a portion of the recording for review.

The computer implemented method may be performed by a computer as part of a computer application (or “app”). As described in more detail below, the computer implemented method may be performed by a processor acting as part of a personal computing assistant.

The patient interaction may comprise any verbal, physical, or other interaction between a patient and a clinical professional (e.g. a doctor, pharmacist, medical specialist, medical worker, medical secretary etc.) The patient interaction may take place in person or remotely, for example, over a video connection.

The recording may comprise any record of a patient interaction with a clinical professional. For example, the recording may comprise an audio recording of the patient interaction, a visual recording of the patient interaction, an audiovisual recording (e.g. a video), images of the patient and/or a written recording (e.g. a recording in text form) of a patient interaction with a clinical professional. The recording may comprise annotations (e.g. annotations or notes made by the clinical professional). In some embodiments the recording may further comprise attachments, such as, for example, images or video clips that were shown to the patient by the clinical professional during the interaction.

Generally, the recording of the patient may be time-stamped in a manner such that different parts of the recording may be associated with a different periods of time (e.g. relative to the absolute clock time, or relative to the start of the recording).

In some embodiments, the block 102 in FIG. 1 may comprise accessing a pre-recorded recording of the patient interaction with a clinical professional. For example, stored in a database of recordings of patient interactions.

In some embodiments, the block 102 in FIG. 1 may comprise making a recording of a patient interaction with a clinical professional. For example, block 102 may comprise, using a recording device, (e.g. such as video or audio recording equipment) to record the patient interaction with the clinical professional.

In block 104, the method 100 comprises acquiring physical data relating to a physical state of the patient during the recording.

Generally, the physical data may comprise any type of data that may be used to gain an insight into the emotional state of the patient (such that it may be determined or inferred whether the patient has understood or is in an emotional state to be able to understand the clinical professional).

For example, in some embodiments, the physical data may comprise postural data (e.g. information relating to the posture of the patient). In some embodiments, the physical data may comprise physiological measurements. Postural data may indicate a patient's emotional state, for example, depending on how the patient holds themselves, the direction the patient faces during the interaction, whether they look at the clinical professional or stare away in the distance. Physiological measurements may provide an insight into the emotional state of the patient via the patient's physical response to the contents of the interaction with the clinical professional.

Examples of physiological measurements that may be used as physical data include, but are not limited to, one or more of (e.g. any one or any combination of ones of) vital sign measurements such as heart-rate or blood pressure measurements, cortisol measurements, skin conductance measurements, Electroencephalography (EEG) measurements, Electromyography (EMG) measurements, or Electrocardiogram (ECG) measurements. Such measurements may be used to determine that the patient is stressed (e.g. the patient's physical state is one of stress), excited or otherwise emotional and therefore that the patient may be unable to fully take in or understand what is being said to them.

Examples of postural data include, but are not limited to, one or more of (e.g. any one or any combination of ones of) data relating to a facial expression of the patient, a seating position of the patient, a pupil dilation of the patient, speech data and eye gaze movements of the patient. Such postural data may be used to determine, for example, where the patient is looking during the patient interaction, e.g. whether they are looking at or away from or facing or facing away from the clinical professional. Speech data may include, for example the tone, or pitch of the patient's voice, number or duration of hesitations, whether the response is coherent or not (e.g. whether the patient uses incorrect words) and/or whether the patient repeats certain words or phrases. Such insights may indicate whether the patient is listening and/or able to take in and understand what is being said to them.

As noted above, the physical data relates to a physical state of the patient during the recording. Thus, the physical data may comprise physical data taken (e.g. recorded or measured) during a time period that overlaps with, is encompassed by, or is contemporaneous with (or approximately contemporaneous with) the time period in which the recording of the patient interaction with the clinical professional was made.

Generally the physical data may be time-stamped or recorded in such a way that portions of physical data may be matched to corresponding portions of the recording (e.g. to portions of the recording that occurred at the same time, or approximately the same time, as the physical data was taken).

In some embodiments, block 104 of FIG. 1 may comprise accessing stored physical data. For example, data stored in a database of physical data (and corresponding recordings of patient interactions).

In some embodiments, block 104 of FIG. 1 may comprise taking measurements or otherwise recording the physical data. For example, block 104 may comprise acquiring the physical data using medical equipment (e.g. such as a heart rate monitor, blood pressure measuring device or other medical device) or any other suitable device.

In some embodiments, the physical data may be derived from the recording of the patient interaction with the clinical professional. For example, the recording may comprise a video recording of the patient and physical data such as postural data (such as seating position, eye-gaze, or any other postural measurement) may be derived from the recording.

Turning now to block 106 of FIG. 1, in some embodiments, flagging a portion of the recording for review based on the physical data, may comprise flagging a portion of the recording corresponding to a duration of time in which the physical data is consistent with the patient being emotional. In this sense, the physical data may be consistent with (e.g. indicative of or possibly indicative of) the patient being emotional if the physical data suggests the patient is distracted or exhibiting emotion such as agitation, stress, shock, and is thus not paying attention (or only paying reduced attention) to the interaction. Generally, a portion of the recording may be flagged for review if the physical data is consistent with the patient being in an emotional state which makes processing information more difficult for the patient. In some embodiments, a portion of the recording may be flagged for review if the physical data is consistent with the patient not having understood the information provided to them.

In some embodiments, the method 100 may comprise flagging a portion of the recording corresponding to a duration of time in which the physical data is outlying compared to baseline physical data. Outlying physical data may indicate, for example, that the patient is emotional and exhibiting a physical response to the emotion.

Baseline physical data may comprise physical data collected whilst the patient is in a normal, e.g. non-distracted (or attentive) state. For example, the baseline physical data may be collected during a time period in which the patient is relaxed, or collected before, or at the beginning of the patient interaction (e.g. before the patient becomes emotional and/or distracted or agitated etc.) Physical data may be outlying compared to the baseline physical data if it is statistically outlying (e.g. raised or lowered by more than a threshold percentage or threshold number of standard deviations) or if there is a sudden change or spike in the physical measurement (e.g. sudden raised vital sign measurements). The skilled person will appreciate that these are examples of how to determine outlying data and will be familiar with other examples of how to determine outlying data.

In some embodiments, a portion of the recording may be flagged based on postural data, for example, a facial expression, posture and/or gaze pattern observed in a corresponding portion of the physical data. For example, eye tracking may indicate visual attention (for example, gazing in a different direction to the clinical professional or concentrated gaze at the clinical professional may indicate distraction). Furthermore, pupil dilation and/or constriction may correspond to emotional arousal and/or cognitive load. Vital signs may provide insights into emotional state, for example, EEG, EMG and/or ECG data. In some embodiments, EEG may be measured using a head-mounted wearable sensor. Cortisol detection and/or skin conductance may be measured using a body wearable sensor.

Generally, if high arousal is determined (e.g. sudden, outlying, or raised vital sign measurements, raised cortisol measurements, increased electrical activity in the brain via Electroencephalography measurements), the patient is likely emotional and therefore not listening. They may not be listening both at the moment in time of the arousal and/or during a time period subsequent to the arousal.

In addition to the embodiments above where portions of the recording are flagged based on the physical data, in some embodiments, portions of the recording may further be flagged based on other factors or criteria.

For example, in some embodiments selected durations of time corresponding to certain topics discussed in the patient interaction may be flagged for review. For example, in some embodiments, the method 100 may further comprise analyzing the recording of the patient interaction to determine one or more topics related to the content of the interaction. The block 106 of flagging a portion of the recording for review, may further be based on the determined one or more topics. In this way, it may be possible to only flag those topics that are important for review, for example those topics that are relevant to the patient's clinical condition.

The skilled person will be familiar with methods for analyzing content such as a recording of a patient interaction to determine the topics (e.g. subjects) covered by the recording. For example, natural language processing may be used to (automatically) generate summaries of a document or transcript of an audio recording. The skilled person will further be familiar with other content tagging methods such as the automatic tagging of video data based on content and/or methods for automatically generating a table of contents for a document or transcript of a recording. Such methods may be used to analyses a recording of a patient interaction and determine one or more topics related to the content of the interaction.

As noted above, flagging a portion of the recording for review may be further based on the determined one or more topics. For example, in some embodiments, the method 100 may comprise matching the determined one or more topics to a predefined list of topics. The block 106 of flagging a portion of the recording for review may then further comprise flagging a portion of the recording based on the matching.

A predefined list of topics may comprise, for example, a list of clinical conditions. Such a list of conditions may be a generic list of medical conditions, or a list of conditions associated with the patient (for example, extracted from their medical records). In some embodiments, only those topics matching topics on such a predefined list may be flagged for review.

In some embodiments, the method may further comprise ranking the determined one or more topics according to importance. The block 106 of flagging a portion of the recording for review, may further comprise flagging a portion of the recording based on the ranking A ranking may be determined, for example, based on the severity of the topics. In some embodiments, for example, only the top 2 or 3 topics may be flagged for review at any one time. This may ensure that a manageable number of topics are flagged for review (and thus prevent overloading the patient further).

As well as, or alternatively to flagging portions of data based on the topics or content of the recording of the patient interaction, other data may be captured and analyzed so as to provide useful information to the patient after an interaction with a clinical professional.

For example, according to some embodiments, the method 100 may further comprise acquiring further audio data during a time period subsequent to the recording, analyzing the further audio data to determine one or more topics related to the content of the further audio data. Flagging a portion of the recording for review may further be based on the determined one or more topics related to the content of the further audio data.

Further audio data may comprise, for example a recording of a conversation between the patient and another individual, such as a friend, caregiver or relative.

Analyzing the further audio data may comprise, for example, determining one or more topics related to the content of the further audio data using any of the techniques described above for determining the contents of a recording, such as natural language processing, and/or automated generation of document summaries or tables of contents.

Flagging a portion of the recording for review based on the topics of the content of the further audio data may comprise, for example, matching a topic of the further audio data to a topic of the patient interaction with a clinical professional. In this way, if in a subsequent conversation the patient says, that they didn't understand one or more of the topics discussed in the interaction with the clinical professional, then these topics may be flagged for review. Portion(s) may further be flagged based on whether the patient subsequently mentions the topic. For example, not mentioning a topic may indicate that one or more aspects of the topic have been overlooked. In this way, the patient's subsequent conversations may be analyzed in order to provide them with the most relevant information in order to help them better understand their condition.

In some embodiments, topics may alternatively or additionally be flagged according to the frequency with which the patient subsequently discusses the topic in subsequent conversations. For example, if the patient mentions “chemotherapy” more often than “melanoma” then the patient may require more information on chemotherapy. Thus, in some embodiments, the method 100 may comprise determining a frequency with which each of the one or more topics appears in the further audio data. Flagging 106 a portion of the recording for review may then be further based on the determined frequencies.

In some embodiments, flagging a portion of the recording 106 may alternatively or additionally be based on the patient's internet history. For example, if subsequent to a patient interaction with a clinical professional, the patient repeatedly searches the internet for particular things (e.g. cancer prognosis, hair loss etc.) then this may mean that the patient did not understand, or needs additional information relating to these topics. Thus, portions of the recording of the patient interaction with the clinical professional may be flagged based on the subsequent internet search history of the patient.

As such, in some embodiments the method 100 may further comprise acquiring details of the patient's internet search history during a time period subsequent to the recording, and analyzing the internet search history to determine one or more topics related to the content of internet search history. Flagging 106 a portion of the recording for review may then further be based on the determined one or more topics related to the content of the internet search history.

Once one or more portions of the recording are flagged for review (e.g. according to any of the examples above), the method 100 may further comprise acquiring further physical data relating to a physical state of the patient during a time period subsequent to the recording, determining a mental state of the patient, based on the further physical data, and displaying the flagged portion of the recording to the patient.

The further physical data may comprise any of the types of data described above with respect to the physical data. For example, any of the postural or physiological data types, as described above.

The time period subsequent to the recording may comprise any time period after the patient interaction with the clinical professional. For example, a time period when the patient is at home, for example, resting.

Determining a mental state of the patient may comprise, for example, determining from the further physical data that the patient is relaxed, alert or attentive. The mental state may indicate that the patient is ready to review the flagged portions of the patient's interaction with the clinical professional.

The mental state of the patient may be determined based on any of the methods described above with respect to block 102 of method 100. For example, by analyzing postural and/or physiological data comprised in the further physical data. It may be determined that the patient's mental state is relaxed, for example, if the further physical data is consistent with (e.g. not outlying from) baseline data.

The flagged portion(s) of the recording may be displayed to the patient based on the mental state of the patient, for example, by only displaying the flagged portions to the patient during time periods in which the further physical data is consistent with the patient being relaxed, attentive or otherwise able to concentrate on the information in the flagged portions of recording.

The flagged portions of the recording may be displayed to the patient, for example, on a display device, such as a computer screen, computer tablet, phone screen, speakers, or any other device for displaying or relaying the flagged portion(s) of the recording.

In this way, further physical data may be used to determine a time period where the patient may be able to concentrate on and understand the flagged portions of data.

In some embodiments, additionally or alternatively, the method 100 may comprise alerting the clinical professional or another caregiver (such as a nurse, volunteer, health coach or informal caregiver) to the flagged portion of the recording. In this way, the clinical professional may be alerted to potential topics that may need more attention in subsequent appointments with the patient. It may be indicated to the clinical professional which topics (e.g. items of the dialog) should be re-discussed. The emotional response of the patient to these items may also be indicated in order to guide the clinical professional on the best way to approach the patient.

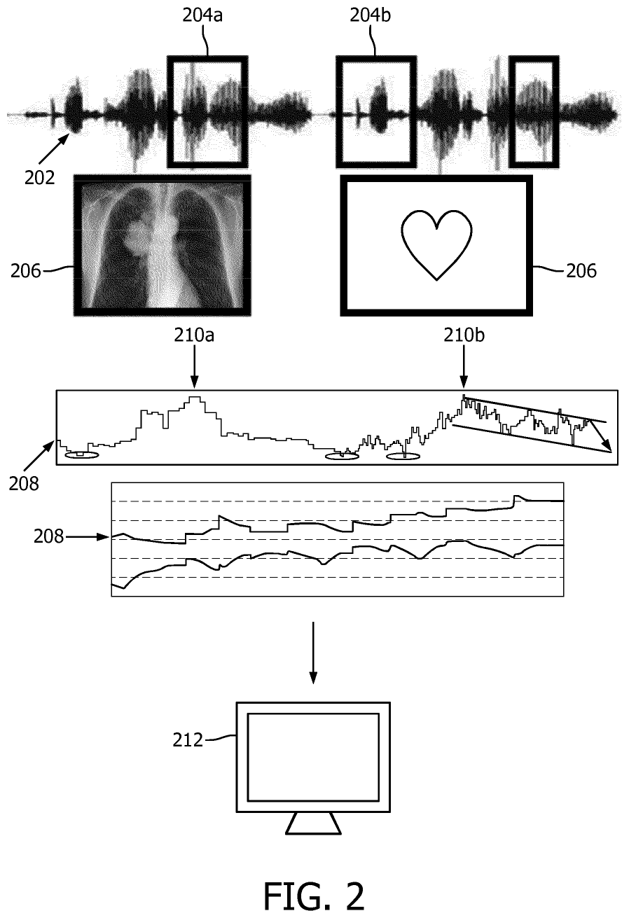

Turning now to FIG. 2, FIG. 2 illustrates an example of the method 100 according to some embodiments herein. A recording 202 of a patient interaction with a clinical professional is acquired. In this embodiment, the recording comprises an audio recording of a patient interaction with a clinical professional. The recording may comprise one or more additional pieces of content 206, for example, videos or photographs that were shown to the patient by the clinical professional during the patient interaction with the clinical professional. Acquiring a recording of a patient interaction with a clinical professional was described with respect to block 102 above and the details will be understood to apply equally to the embodiment of FIG. 2.

Physical data 208 is also acquired. In this example, the physical data comprises heart rate measurements (top panel) and eye gaze measurements (lower panel of 208). The physical data is analyzed to determine durations of time in which the physical data is outlying compared to baseline physical data. The physical data is determined to be outlying at points 210a and 210b and these time intervals are matched to the corresponding portions 204a and 204b of the recording 202 of the patient interaction. Based on the durations of time in which the physical measurements were outlying, portions 204a and 204b are then flagged for review.

Flagging a portion of a recording for review, based on physical data was discussed above with respect to block 106 and the details therein will be understood to apply equally to the embodiment of FIG. 2.

At a subsequent time, for example, when further physical data indicates that the patient is relaxed, the flagged portions 204a and 204b may be displayed to the patient, for example, on an electronic screen or display 212 for review. In this way, the patient may be able to review, in a relaxed way, portions of an interaction with a medical professional that they may otherwise not have understood or been able to take in. Thus the patient's understanding of their condition may be improved.

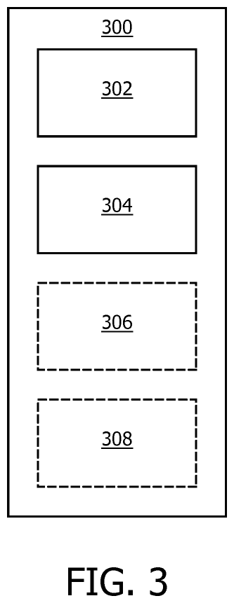

Turning now to FIG. 3, in some embodiments, there is a system 300 configured for flagging a portion of a recording of a patient interaction with a medical professional for review. The system 300 comprises a memory 304 comprising instruction data representing a set of instructions. The system 300 further comprises a processor 302 configured to communicate with the memory 304 and to execute the set of instructions. The set of instructions when executed by the processor may cause the processor to perform any of the embodiments of the method 100 as described above. The memory 304 may be configured to store the instruction data in the form of program code that can be executed by the processor 302 to perform the method 100 described above.

In some implementations, the instruction data can comprise a plurality of software and/or hardware modules that are each configured to perform, or are for performing, individual or multiple steps of the method described herein. In some embodiments, the memory 304 may be part of a device that also comprises one or more other components of the system 300 (for example, the processor 302 and/or one or more other components of the system 300). In alternative embodiments, the memory 304 may be part of a separate device to the other components of the system 300.

In some embodiments, the memory 304 may comprise a plurality of sub-memories, each sub-memory being capable of storing a piece of instruction data. In some embodiments where the memory 304 comprises a plurality of sub-memories, instruction data representing the set of instructions may be stored at a single sub-memory. In other embodiments where the memory 304 comprises a plurality of sub-memories, instruction data representing the set of instructions may be stored at multiple sub-memories. Thus, according to some embodiments, the instruction data representing different instructions may be stored at one or more different locations in the system 300. In some embodiments, the memory 304 may be used to store information, such as the recording of the patient interaction, the physical data or other data relevant to determinations made by the processor 302 of the system 300 or from any other components of the system 300.

The processor 302 can comprise one or more processors, processing units, multi-core processors and/or modules that are configured or programmed to control the system 300 in the manner described herein. In some implementations, for example, the processor 302 may comprise a plurality of (for example, interoperated) processors, processing units, multi-core processors and/or modules configured for distributed processing. It will be appreciated by a person skilled in the art that such processors, processing units, multi-core processors and/or modules may be located in different locations and may perform different steps and/or different parts of a single step of the method described herein.

Briefly, the set of instructions, when executed by the processor 302, cause the processor 302 to acquire a recording of a patient interaction with a clinical professional and acquire physical data relating to a physical state of the patient during the recording. The set of instructions, when executed by the processor 302, further cause the processor to flag a portion of the recording for review, based on the physical data.

Acquiring a recording of a patient interaction, acquiring physical data and flagging a portion of the recording for review were all described above with respect to the method 100 and the details therein will be understood to apply equally to the system 300.

In some embodiments, the system 300 may be, or may comprise part of a digital personal assistant. In some embodiments, the system 300 may be or may comprise part of a device such as a smart watch, activity monitor or smartphone.

It will be appreciated that the system 300 may comprise additional components to those illustrated in FIG. 3. For example, in some embodiments the system 300 may comprise one or more recording devices 306 for recording the patient interaction with the clinical professional. The recording device 306 may comprise, for example, a camera, microphone or other video recording equipment. The recording device 306 may be configured to acquire (e.g. record) the recording of the patient interaction, as described above with respect to block 102 of method 100.

In some embodiments, the system 300 may additionally comprise one or more devices 308 for acquiring the physical data. For example, the system 300 may comprise one or more pieces of equipment for making physiological measurements such as vital sign measurements. The device 308 may be configured to acquire the physical data as described above with respect to block 104 of method 100.

The system 300 may further comprise one or more communication interfaces for receiving and/or sending information, for example, to and from a database. The system 300 may further comprise one or more user interfaces such as a display screen, mouse, keyboard or any other user interface allowing information to be displayed to a user or input to be received from a user. For example, such a display may be used to display the flagged portion of the recording to the patient, as described above. In some embodiments, the system 300 may further comprise a power source such as a battery or mains power connection.

According to further embodiments, there is a computer program product comprising a non-transitory computer readable medium, the computer readable medium having computer readable code embodied therein, the computer readable code being configured such that, on execution by a suitable computer or processor, the computer or processor is caused to perform the method 100.

In some embodiments, the computer program product may comprise an app (“application”), such as an app for a computer, tablet or mobile phone. In some embodiments, the computer program product may be comprised in (e.g. installed on or part of) a personal digital organizer or digital assistant (e.g. such as a “virtual doctor”).

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer implemented method comprising:
acquiring a recording of a patient interaction with a clinical professional;
acquiring physical data relating to a physical state of the patient during the recording; and
based on the physical data, flagging a portion of the recording for review.

2. A method as in claim 1 wherein flagging a portion of the recording comprises:
flagging a portion of the recording corresponding to a duration of time in which the physical data is consistent with the patient being emotional.

3. A method as in claim 1 wherein flagging a portion of the recording for review comprises:
flagging a portion of the recording corresponding to a duration of time in which the physical data is outlying compared to baseline physical data.

4. A method as claim 1 wherein the physical data comprises postural data or physiological measurements.

5. A method as in claim 4 wherein the postural data or physiological measurements comprise one or more of:
vital sign measurements;
heart-rate measurements;
cortisol measurements;
skin conductance measurements;
Electroencephalography, EEG, Electromyography, EMG, or Electrocardiogram, ECG measurements;
a facial expression of the patient;
a seating position of the patient;
a pupil dilation of the patient;
speech data; and
eye gaze movements of the patient.

6. A method as in claim 1 further comprising:
analyzing the recording of the patient interaction to determine one or more topics related to the content of the interaction; and
wherein flagging a portion of the recording for review is further based on the determined one or more topics.

7. A method as in claim 6 further comprising:
ranking the determined one or more topics according to importance; and
wherein flagging a portion of the recording for review further comprises flagging a portion of the recording based on the ranking.

8. A method as in claim 6 further comprising:
matching the determined one or more topics to a predefined list of topics; and
wherein flagging a portion of the recording for review further comprises flagging a portion of the recording based on the matching.

9. A method as in claim 1 further comprising:
acquiring further audio data during a time period subsequent to the recording;
analyzing the further audio data to determine one or more topics related to the content of the further audio data; and
wherein flagging a portion of the recording for review is further based on the determined one or more topics related to the content of the further audio data.

10. A method as in claim 9 further comprising:
determining a frequency with which each of the one or more topics appears in the further audio data; and
wherein flagging a portion of the recording for review is further based on the determined frequencies.

11. A method as in claim 1 further comprising:
acquiring details of the patient's internet search history during a time period subsequent to the recording;
analyzing the internet search history to determine one or more topics related to the content of internet search history; and
wherein flagging a portion of the recording for review is further based on the determined one or more topics related to the content of the internet search history.

12. A method as in claim 1 further comprising:
acquiring further physical data relating to a physical state of the patient during a time period subsequent to the recording;
determining a mental state of the patient, based on the further physical data; and
displaying the flagged portion of the recording to the patient, based on the mental state.

13. A method as in claim 1 further comprising:
alerting the clinical professional or another caregiver to the flagged portion of the recording.

14. A computer program product comprising a non-transitory computer readable medium, the computer readable medium having computer readable code embodied therein, the computer readable code being configured such that, on execution by a suitable computer or processor, the computer or processor is caused to perform the method of claim 1.

15. A system comprising:
a memory comprising instruction data representing a set of instructions;
a processor configured to communicate with the memory and to execute the set of instructions, wherein the set of instructions, when executed by the processor, cause the processor to:
acquire a recording of a patient interaction with a clinical professional;
acquire physical data relating to a physical state of the patient during the recording; and
based on the physical data, flag a portion of the recording for review.
